(19) United States
(12) Patent Application Publication
(10) Pub. No.: US 2012/0251595 A1
(43) Pub. Date: Oct. 4, 2012

(54) EMULSION TEMPLATE METHOD TO FORM SMALL PARTICLES OF HYDROPHOBIC AGENTS WITH SURFACE ENRICHED HYDROPHILICITY BY ULTRA RAPID FREEZING

(75) Inventors: Robert O. Williams, III, Austin, TX (US); Keat Theng Chow, Singapore (SG)

(73) Assignee: BOARD OF REGENTS, THE UNIVERSITY OF TEXAS SYSTEM, Austin, TX (US)

(21) Appl. No.: 13/508,931

(22) PCT Filed: Nov. 9, 2010
(86) PCT No.: PCT/US10/56030

§ 371 Date: Jun. 20, 2012

Related U.S. Application Data
(60) Provisional application No. 61/259,237, filed on Nov. 9, 2009.

(51) Int. Cl. B29B 9/12; A61K 9/14; A61P 31/10; A61K 31/496

(52) U.S. Cl. 424/400; 514/254.07; 264/5; 264/9

(57) ABSTRACT

The present invention relates to methods and compositions to prepare small size particles of poorly water soluble agents or drugs with surface enriched hydrophilicity.

### TECHNICAL FIELD OF THE INVENTION

The present invention relates in general to the field of preparing small particles of poorly water soluble agents or drugs, and more particularly, to using fine emulsion templating followed by thin film freezing to form small particles of hydrophobic agents with surface enriched hydrophilicity.

### BACKGROUND ART

Without limiting the scope of the invention, its background is described in connection with the preparation of small particles of poorly water soluble agents or drugs. More specifically, the present invention relates to using an emulsion template followed by ultra-rapid freezing (URF; thin film freezing) to enhance the solubility of poorly water soluble agents via the formation of small particles of hydrophobic agents with surface enriched hydrophilicity.

Poorly water soluble compounds are common among new chemical entities being investigated for therapeutic activity as active pharmaceutical ingredients. High bioavailability and short dissolution times are desirable attributes of a pharmaceutical end product. Bioavailability is a term meaning the degree to which a pharmaceutical product, or drug, becomes available to the target tissue after being administered to the body. Poor bioavailability is a significant problem encountered in the development of pharmaceutical compositions, particularly those containing an active ingredient that is poorly soluble in water. For example, upon oral administration, poorly water soluble drugs tend to be eliminated from the gastrointestinal tract before being absorbed into the circulation.

Oil/Water (O/W) emulsions are frequently used in the pharmaceutical industry to enhance the overall concentration of poorly water soluble and insoluble drugs, due to the high solubility of the active pharmaceutical ingredient in the dispersed oil phase. However, emulsion stability is a concern. Over time, emulsions often coalesce and settle. Additionally, the large volume of the oil and aqueous phases limits the overall drug concentration and yield. To overcome these inherent disadvantages, solvents are often removed from emulsion formulations by lyophilization. It is well recognized that extreme temperature fluctuation such as freezing can result in an increased oil droplet size, leading to physical instability, i.e., aggregation, coalescence and ultimate separation. It has been found that freezing emulsions has resulted in phase separation and destabilization of the active pharmaceutical ingredients, and that dry emulsions did not produce the same degree of dissolution enhancement as was achieved prior to lyophilization. For oral delivery, it is desirable to produce dry powders by lyophilization with high dissolution rates.

It is known that the rate of dissolution of a particulate drug can increase with increasing surface area, i.e., decreasing particle size. Consequently, efforts have been made to control the size and size range of drug particles in pharmaceutical compositions. Current micronization technologies do not produce particles having a surface excess of hydrophilic agent to aid in wetting, dissolution, and bioavailability. It would be an advantage in the art of particle engineering for the pharmaceutical industry to provide a process which resulted in the formation of small particles with a surface layer composed of much more hydrophilic agent. Some efforts aimed at modifying particle structures rely on freezing materials by spraying those materials into a cryogenic liquid. However, these technologies have problems associated with recovering the particles from the cryogenic liquid, handling the cryogenic liquid, and environmental issues.

### DISCLOSURE OF THE INVENTION

The present invention includes a method combining an improved template emulsion method with Ultra Rapid Freezing (URF; thin film freezing; TFF), and compositions resulting from the application of that method. A hydrophobic, poorly water soluble agent, such as an active pharmaceutical ingredient (or a nutraceutical, agricultural, or veterinary product) is prepared in an emulsion (single emulsion or multiple emulsion) that is capable of remaining as an emulsion during application to the cryogenic surface of the thin film freezing apparatus, with a hydrophilic excipient, such as a surfactant or hydrophilic polymer, chosen such that when the emulsion is processed by thin film freezing, after the frozen solvent is removed, the resulting powder is surface enriched such that the active composition displays a surface excess (e.g., greater than about 2%) of the hydrophilic excipient by X-ray photoelectron spectroscopy or another suitable method that measures surface excess of hydrophilic agent. In essence, the hydrophobic, poorly water soluble agent is now rendered hydrophilic due to this surface excess of hydrophilic excipient. Thus, this invention allows the formulation of BCS Class II and IV drugs to be formulated into bioavailable dosage forms.

In one aspect, the present invention includes a method of making particles with surface enriched hydrophilicity by template emulsion. This method comprises the steps of (i) dissolving or dispersing one or more hydrophobic agents in an effective amount of an organic solvent and an emulsifying agent (e.g., surfactant, emulsion stabilizer (e.g., hydrophilic polymer) or other agents capable of providing a surface excess), wherein the one or more agents and the solvent form an organic phase mixture, (ii) homogenizing the organic phase mixture with an aqueous phase mixture to form a template emulsion, and (iii) cryogenically processing droplets of the template emulsion by ultra rapid freezing under conditions that do not trigger a Liedenfrost effect during the freezing process to produce frozen emulsion particles.

The template emulsion drops are normally frozen such that the droplet freezes in less than about 10 seconds, about 5 seconds, about 1 second or about 0.5 seconds, when contacting the cryogenic surface, depending on the solvent chosen. The method may further comprise the steps of collecting the frozen emulsion particles and drying the frozen emulsion particles, the resulting product being a dry powder that is surface enriched for the hydrophilic excipient over the agent. The frozen emulsion particles may be collected in liquid nitrogen, after which they may also be dried by lyophilization. The template emulsion may be a single emulsion or a multiple emulsion. In one embodiment of the invention, the template emulsion is capable of remaining as an emulsion during application to the cryogenic surface of the thin film freezing apparatus. Extreme temperature fluctuation such as freezing can result in an increased oil droplet size, leading to physical instability, i.e., aggregation, coalescence and ultimate separation.

The admixture of organic and aqueous phase mixtures may be homogenized by high-shearing, using a technique such as ultrasonication. Ultrasonication may be performed using a probe sonicator. The mean size of the resulting emulsion droplets may be approximately 270 to 300 nm.

The organic solvent in the organic phase mixture used by this method may comprise one or more organic compounds and one or more emulsifying agents. These organic compounds are defined further as organic solvents that are not miscible with a continuous external phase of the template emulsion. In one embodiment of the present invention, one of the organic solvents used in the organic phase is chloroform, and the concentration of chloroform used is about 20% v/v. One of the emulsifying agents in the organic phase may be lecithin. The organic phase mixture may comprise an oil.

The aqueous phase mixture used by this method may comprise one or more polar solvents not miscible with the organic phase and one or more excipients. In one embodiment of the present invention, one of the polar solvents in the aqueous phase mixture is water, and the concentration of water used is about 80% v/v. The one or more excipients in the aqueous phase mixture may comprise at least one of a hydrophilic polymer, such polyvinyl pyrrolidone (PVP), polyvinyl alcohol (PVA) or hydroxypropyl methylcellulose (HPMC), and an emulsifying agent. One or more of the excipients in the aqueous phase mixture may be a surfactant or a hydrophilic polymer.

The agent or agents used by this method may comprise an active pharmaceutical agent. The active pharmaceutical agent used in this method may be a Biopharmaceutical Classification System (BCS) Class II or Class IV drug. Also, the agent or agents used by this method may be hydrophobic or poorly soluble in water. The method's applicability is not limited to pharmaceutical agents, and it may be applied to nutraceutical, agricultural, or veterinary products.

In one embodiment of the invention, the powder resulting from drying the frozen emulsion particles is surface enriched such that the active composition displays a surface excess of the one or more hydrophilic excipient by X-ray photoelectron spectroscopy or another suitable method that measures surface excess of the one or more agents. The surface excess may be greater than about 2%.

In another aspect, the present invention includes compositions made by a process comprising the steps of (i) dissolving or dispersing one or more hydrophobic agents in an effective amount of an organic solvent and an emulsifying agent, wherein the one or more agents and the solvent form an organic phase mixture; (ii) homogenizing the organic phase mixture with an aqueous phase mixture, to form a template emulsion; and (iii) cryogenically processing droplets of the template emulsion by ultra rapid freezing under conditions that do not trigger a Liedenfrost effect during the freezing process to produce frozen emulsion particles.

The template emulsion drops used to generate the composition are normally frozen such that the droplet freezes in less than about 10 seconds, about 5 seconds, about 1 second or about 0.5 seconds, when contacting the cryogenic surface. The process used to make the composition may further comprise the steps of collecting the frozen emulsion particles and drying the frozen emulsion particles, the resulting product being a dry powder that is surface enriched for the hydrophilic excipient over the agent. The process used to make the composition may include the step of collecting frozen emulsion particles in liquid nitrogen, after which they may also be dried by lyophilization. The template emulsion used in the process may be a single emulsion or a multiple emulsion. In one embodiment of the invention, the template emulsion used in the process is capable of remaining as an emulsion during application to the cryogenic surface of the thin film freezing apparatus.

The admixture of organic and aqueous phase mixtures used in the process used to prepare the composition may be homogenized by high-shearing, using a technique such as ultrasonication. Ultrasonication may be performed using a probe sonicator. The mean size of the resulting emulsion droplets may be approximately 270 to 300 nm.

The organic solvent in the organic phase mixture used in the process may comprise one or more organic compounds and one or more emulsifying agents. These organic compounds are defined further as organic solvents that are not miscible with a continuous external phase of the template emulsion. In one embodiment of the present invention, one of the organic solvents used in the organic phase is chloroform, and the concentration of chloroform used is about 20% v/v. One of the emulsifying agents in the organic phase may be lecithin. The organic phase mixture may comprise an oil.

The aqueous phase mixture used in the process to produce the claimed composition may comprise one or more polar solvents and one or more excipients. In one embodiment of the present invention, one of the polar solvents in the aqueous phase mixture used in the process is water, and the concentration of water used is about 80% v/v. The one or more excipients in the aqueous phase mixture may comprise at least one of a hydrophilic polymer, such polyvinyl pyrrolidone (PVP), polyvinyl alcohol (PVA) or hydroxypropyl methylcellulose (HPMC), and an emulsifying agent. One or more of the excipients in the aqueous phase mixture may be a surfactant or a hydrophilic polymer.

The agent or agents used in the process may comprise an active pharmaceutical agent. The active pharmaceutical agent may be a Biopharmaceutical Classification System (BCS) Class II or Class IV drug. Also, the agent or agents used in the process may be hydrophobic or poorly soluble in water. The process's applicability is not limited to producing compositions containing pharmaceutical agents; it may also be applied to nutraceutical, agricultural, or veterinary products.

In one embodiment of the invention, the composition resulting from drying the frozen emulsion particles is surface enriched such that the active composition displays a surface excess of the one or more hydrophilic excipient by X-ray photoelectron spectroscopy or another suitable method that measures surface excess of the one or more agents. The surface excess may be greater than about 2%.

In another embodiment of the present invention, the composition would further comprise a pharmaceutically acceptable carrier.

Another embodiment of the present invention is a composition comprising a heterogenous lyophilized particle comprising a hydrophilic polymer having an inner portion enriched with an active ingredient and surrounded by a surface portion having a surface excess of surfactant made from a rapidly frozen homogenous solution of a template emulsion. The homogenous solution may be rapidly frozen by ultra rapid freezing (URF). The invention also includes a non-encapsulated particle comprising a heterogenous lyophilized particle which comprises a hydrophilic polymer having an inner portion enriched with an active ingredient and surrounded by a surface portion having a surface excess of surfactant made from a rapidly frozen homogenous solution of a template emulsion. The non-encapsulated particle may be produced by rapidly freezing the homogeneous solution by ultra rapid freezing (URF).

The present invention also includes a particle comprising a heterogenous lyophilized hydrophilic polymer particle, the particle comprising an inner portion enriched with an active ingredient over a surfactant and surrounded by a surface portion having a surface excess of surfactant over active agent made from a rapidly frozen homogenous solution of a template emulsion by a suitable cryogenic technique such as ultra rapid freezing (URF).

### DESCRIPTION OF THE DRAWINGS

For a more complete understanding of the features and advantages of the present invention, reference is now made to the detailed description of the invention along with the accompanying figures and in which:

FIG. 1 shows the sample preparation process for O/W template emulsions (left panel) and co-solvent mixtures (right panel) for URF (Ultra Rapid Freezing, or Thin Film Freezing).

FIG. 2 illustrates the processing of the samples (either O/W template emulsions or co-solvent mixtures) by URF, as well as the composition of the dry powders resulting from collecting and lyophilizing the frozen particles resulting from URF processing.

FIG. 3 shows the droplet size of template emulsions containing the hydrophilic excipients polyvinyl pyrrolidone Plasdone® K17 (PVP), polyvinyl alcohol (PVA), and hydroxypropyl methylcellulose E5 (HPMC). Mean emulsion droplet size was about 270-300 nm.

FIG. 4 shows scanning electron micrographs of dry powders resulting from URF processing of template emulsion system samples (ITZ:lecithin:PVP and ITZ:lecithin:PVA) and co-solvent system samples (ITZ:lecithin:PVA). Three levels of magnification (10×, 50×, and 100×) are shown for each sample.

FIG. 5 shows X-ray diffractograms of (i) bulk ITZ, (ii) ITZ physically mixed with lecithin and hydrophilic excipients, and (iii) URF powders resulting from processing emulsion template samples and co-solvent system samples containing ITZ, lecithin, and hydrophilic excipients.

FIG. 6 shows surface excess analysis resulting from X-ray photoelectron spectroscopy analysis of O/W emulsion template ITZ samples (EM) and control formulations consisting of ITZ and the hydrophilic excipients PVP, HPMC, and PVA in a co-solvent system (SOL).

FIG. 7 shows supersaturated dissolution testing dissolution profiles of (a) O/W emulsion template ITZ samples (EM), and (b) co-solvent system ITZ samples (SOL) containing the hydrophilic excipients PVP, HPMC, and PVA. Testing was performed at 10× supersaturation. The amount of powders employed in dissolution studies corresponded to 5 mg ITZ.

FIG. 8 shows supersaturated dissolution testing dissolution profiles of (a) O/W emulsion template ITZ samples (EM), and (b) co-solvent system ITZ samples (SOL) containing the hydrophilic excipients PVP, HPMC, and PVA. Testing was performed at 100× supersaturation.

FIG. 9 shows AUDC (Area Under the Dissolution Curve) analysis for (a) O/W emulsion template ITZ samples (EM), and (b) co-solvent system ITZ samples (SOL) containing the hydrophilic excipients PVP, HPMC, and PVA. Testing was performed at 10× supersaturation.

FIG. 10 shows AUDC (Area Under the Dissolution Curve) analysis for (a) O/W emulsion template ITZ samples (EM), and (b) co-solvent system ITZ samples (SOL) containing the hydrophilic excipients PVP, HPMC, and PVA. Testing was performed at 100× supersaturation.

FIG. 11 shows Scanning electron micrographs of powders from EXAMPLE 1 (ITZ:lecithin:PVP=2:1:1) (a), EXAMPLE 2 (ITZ:lecithin:PVA=2:1:1) (b), EXAMPLE 3 (ITZ:lecithin:HPMC E5=2:1:1) (c), and EXAMPLE 4 (control formulation, ITZ:lecithin:PVA=2:1:1).

FIG. 12 shows the surface excess of ITZ and lecithin in particles produced from template emulsion (EM) and control formulations consisting of drug and excipients in a co-solvent system (SOL).

FIG. 13 shows dissolution profiles of particles produced from template emulsion (EM) and control formulations (SOL) with ITZ:lecithin:PVA:ext-HPMC E5=2:1:1:0.5 (a), and ITZ:lecithin:PVA:ext-HPMC E50=2:1:1:0.5 (b). The amount of powders employed in dissolution studies corresponded to 50 mg ITZ.

FIG. 14 shows surface excess of ITZ and lecithin in particles produce from template emulsion (EM) with high ITZ potency and control formulations consisting of drug and excipients in a co-solvent system (SOL).

FIG. 15 shows dissolution profiles of particles produced from template emulsion (EM) with high ITZ potency and control formulations (SOL) with ITZ:lecithin:PVA (a), and ITZ:lecithin:PVA:ext-HPMC E5 (b). The amount of powders employed in dissolution studies corresponded to 50 mg ITZ.

### DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The therapeutic potential of many Active Pharmaceutical Ingredients (APIs), particularly the Biopharmaceutical Classification System class II compounds fails to be maximized due to their poor aqueous solubility Enhancing aqueous solubility of such drugs is essential in order to improve bioavailability, minimize drug dose and toxicity, and improve therapeutic efficacy.

Nanoparticulate systems reduce variability and increase bioavailability of poorly water soluble APIs through enhanced absorption due to improved wetting and dissolution. Hydrophobic APIs are not the only compounds that benefit from delivery as nanoparticulate systems. Oral delivery of proteins, peptides, and nucleic acids has proven exceedingly difficult. While being water soluble, these compounds are susceptible to denaturation post-administration when exposed to low pH and gastric enzymes. Most proteins have poor absorption across the intestinal barrier as well and therefore, micro- and nanoparticulate carrier systems could help increase absorption of these compounds.

One of the simplest methods to manufacture solid nanoparticles is through emulsification. Common emulsification methods such as high shear mixing with a rotor-stator mixer, high pressure homogenization, or sonication are used to prepare either oil-in-water (O/W) or water-in-oil (W/O) emulsions. Emulsifying agents preferentially orient between the two phases at the interface of the droplet to prevent coalescence. Generally, oils or water-immiscible organic solvents and water are the typical solvents. The API is preferentially dissolved in the more soluble of the two phases (i.e. organic or oil phase for poorly water soluble APIs). Particles are formed during evaporation of the solvents either through increased heat and/or reduced pressure depositing the API within the core or adsorbed onto the surface. Mean particle size of the final particles is dependant on the droplet size of the internal phase and can range from nanoparticles to microparticles depending on the method of manufacture. Creating multiple emulsions such as oil-in-water-in-oil (O/W/O) or water-in-oil-in-water (W/O/W) can lead to multiple layers allowing more flexibility and creativity in designing delivery systems according to the specific requirements of the clinical endpoint.

Microemulsions differ from coarse emulsions based on size and method of polymerization and are thermodynamically stable systems. Creation of a microemulsion requires that an emulsion (O/W) be formed in the presence of a co-surfactant, such as lecithin. The microemulsion template technology developed by Mumper et al. utilizes microemulsions as a template for the formation of nanoparticles. The particle size is dependent on the internal droplet size of the microemulsion and since formation of microemulsions leads to a uniform particle size distribution, the resulting nanoparticles are very uniform.

Suitable excipients for the oil phase or the aqueous phase include surfactants, emulsifying agents, and hydrophilic polymers. The skilled artisan would recognize that any excipient that will exist in surface excess over the active agent may be suitable. Suitable emulsion stabilizers include acacia, agar, alginic acid, carrageenan, guar gum, karaya gum, tragacanth, xanthan gum, gelatin, carbomer resins, cellulose ethers, carboxymethyl chitin, peg-n(ethylene oxide) polymer, lays (attapulgite, bentonite, kaolin, magnesium aluminum silicate, microcrystalline) oxides and hydroxides (aluminum hydroxide, magnesium hydroxide, silica) amino acids, peptides, proteins (casein, beta-lactoglobulin), lecithin, phospholipids, and poloxamers.

Suitable surfactants and/or emulsifying agents include alcohol ether sulfates, alkyl sulfates, soaps, sulfosuccinates, quaternary ammonium compounds, alkyl betain derivatives, fatty amine sulfates, difatty alkyl triethanolamine derivatives, lanolin alcohols, polyoxyethylated alkyl phenols, poe fatty amide, poe fatty alcohohl ether, poe fatty amine, poe fatty ester, poloxamers, poe glycol monoethers, polysorbates, and sorbitan esters.

A cryogenic technique, ultra rapid freezing (URF; thin film freezing) has been successfully used for production of amorphous and highly porous nano-structured particles of poorly soluble drugs demonstrating greatly enhanced aqueous solubility and rate of dissolution (Overhoff et al., 2007). URF powders are composed of solid solutions of an API and a polymer stabilizer. The stability of amorphous APIs becomes a concern since crystalline APIs exhibit a lower thermodynamic energy state and are more stable. Amorphous material exhibits a glass transition temperature (T_{g}) which when exposed to temperatures higher than the T_{g}, structural arrangement into a more stable crystalline lattice begins. Therefore, careful attention to particle stability must be given when designing amorphous nanoparticles or microparticles. In order to prevent recrystallization, high T_{g} polymers such as hydroxypropyl methylcellulose (HPMC) or polyvinyl pyrrolidone (PVP) must be included in the composition, preferably intimately mixed within the amorphous composition such as solid dispersion or solid solution. Doing so will increase the overall T_{g} of the composition increasing its physical stability when exposed to higher storage temperatures.

URF involves very rapid freezing (e.g., such that the droplet freezes in less than about 10 seconds, about 5 seconds, about 1 second or about 0.5 seconds, when contacting the cryogenic surface) of droplets of a feed solution containing the API and stabilizing excipients on a cryogenic surface. If the freezing rate is sufficiently fast, phase separation between the API and stabilizing agents is prevented creating molecularly dispersed nanoparticles. Removal of the frozen solvent then follows, yielding high surface area nanoparticles of API in the matrix.

Relative to spray freezing processes that use liquid nitrogen, URF also offers fast heat transfer rates as a result of the intimate and immediate contact between the solution and cold solid surface, but without the complexity of cryogen evaporation (Leidenfrost Effect). The ability to produce amorphous high surface area powders with submicron primary particles with a simple ultra freezing process is of practical interest in particle engineering to increase dissolution rates, and ultimately bioavailability. It is recognized that rapidly exposing the room temperature emulsion to freezing temperatures may destabilize the emulsion.

The Leidenfrost Effect is a phenomenon in which a liquid, in near contact with a mass significantly hotter than the liquid's boiling point, produces an insulating vapor layer which keeps that liquid from boiling rapidly. It is named after Johann Gottlob Leidenfrost, who discussed it in A Tract About Some Qualities of Common Water in 1756.

Previously, criteria for selection of solvents suitable for other fast freezing technologies such as Spray Freezing into Liquid (SFL) included sufficient solubility of the solids and the ability to remove the solvent without re-crystallizing the API. These solvents generally have freezing points between 208K and 273K which are ideal for tray lyophilization. Solvents with freezing points below 208K melt during lyophilization while solvents with freezing points higher than 273K may freeze prematurely within the atomizing nozzle of the SFL apparatus that is submerged below the surface of the liquid cryogen. Because the URF technology applies the droplets directly onto the cryogenic substrate, premature freezing overcomes this and is not a concern and high freezing point solvents may now be used. These solvents could prove beneficial by reducing the lyophilization time or eliminating the solvent removal process altogether as some of these solvents sublime at ambient conditions or higher.

URF feed solutions commonly consist of a dilute solution, often less than 2% by weight, of poorly soluble drug and stabilizing excipients in an aqueous-organic co-solvent system with an optimized solvent ratio. The hydrophobic nature of the drug limits loading and hence, increases organic solvent consumption. Instead of these undesirable dilute solutions, the present invention uses O/W template emulsions (Organic Phase/Water Phase emulsions).

The main advantages of the O/W template emulsions as used as liquid feed solution for URF processing in the present invention are: high drug solubility in the internal oil phase (100% organic solvent) increases loading of poorly soluble drugs; reduced organic solvent requirement; attainment of high concentration of stabilizing excipient with drug molecules due to preferred orientation of excipient/surfactant molecules in the vicinity of oil droplets containing the dissolved drug and thus increased extent of drug stabilization by preventing drug recrystallization; and fine emulsions serve as template for production of micron to submicron particles with high surface area allowing better control of particle size distribution.

The usefulness of O/W template emulsions in the spray freezing into liquid (SFL) process to produce amorphous, micronized powders with enhanced drug solubility has been demonstrated using the model poorly soluble drugs danazol (Rogers et al., 2003) and itraconazole (Chow et al., 2008). The current study extends the emulsion templating approach to the URF process for engineering poorly soluble drugs with greatly enhanced solubilities but with the advantage of not triggering the Liedenfrost effect that is inherent in the SFL process.

This study compares the effectiveness of fine emulsion templating and co-solvent approaches with the URF process to enhance the wetting and solubility of the model drug, itraconazole (ITZ). Itraconazole (ITZ) is a weakly basic broad-spectrum triazole antifungal agent indicated in the treatment of both local and systemic fungal infections; however, successful treatment of infections is often complicated by its low aqueous solubility resulting in variable absorption and plasma concentration. Classified as a BCS class II compound, ITZ has a strongly pH dependent solubility (pKₐ˜3.7) with reported solubilities in acidic and neutral media of approximately 4 μg/mL and 1 ng/mL, respectively. While limited by poor aqueous solubility, the highly lipophilic nature of the compound allows for high permeability of intestinal membranes.

Methods: (1) Sample preparation: O/W template emulsions are generated using two phases, (i) an organic or oil phase, and (ii) an aqueous phase. The organic phase used in the instant study contained itraconazole (ITZ; Hawkins Chemical, Minneapolis, Minn.) 10% w/v in 20% chloroform v/v, plus the emulsifying agent lecithin (Fisher Scientific, Fair Lawn, N.J.). The aqueous phase used in the present study was a solution of a hydrophilic polymer, containing 80% water v/v. The immiscible aqueous and oil phase were homogenized by ultrasonication for 5 minutes using a probe sonicator, to yield an O/W template emulsion.

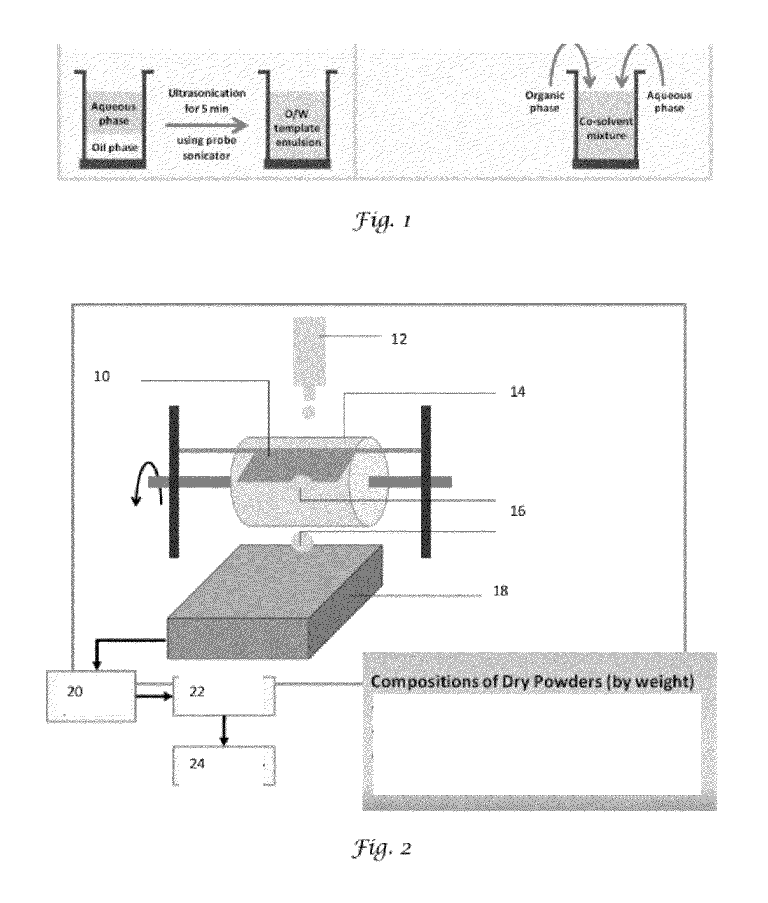

For comparison purposes, a co-solvent system was also used for sample preparation. The co-solvent system was composed of an organic phase (dioxane 65% v/v, ITZ 0.5% w/v in the final co-solvent mixture), and an aqueous phase which were mixed to yield a co-solvent mixture (FIG. 1). FIG. 1 illustrates (left) O/W template emulsions having an organic or ‘oil’ phase and chloroform 20% v/v, ITZ 10% w/v, Lecithin—emulsifying agent with an aqueous phase, water 80% v/v, and Hydrophilic polymer. FIG. 1 illustrates (right) shows a co-solvent system with an organic phase of dioxane 65% v/v and ITZ 0.5% w/v in final co-solvent mixture and an aqueous phase of water 35% v/v, lecithin, and hydrophilic polymer. The hydrophilic polymers used to prepare the samples, such as polyvinyl pyrrolidone PLASDONE® K17 (PVP), polyvinyl alcohol (PVA), and hydroxypropyl methylcellulose E5 (HPMC), function as wetting agents and stabilizing excipients.

(2) Sample processing by URF: Cryogenic technologies have been used to produce highly porous, amorphous, nanostructured particles and microparticles with improved dissolution rates and high supersaturation drug levels relative to the solubility of the crystalline state for poorly water soluble pharmaceutical ingredients. The Spray Freezing into Liquid (SFL) process, which triggers an immediate and distinct insulating vapor layer around the liquid droplet to be frozen (Liedenfrost effect), forms a solid dispersion or solid solution composed of drug domains within a polymer matrix by spraying the drug-excipients solution directly into liquid nitrogen by placing the tip of the nozzle beneath the surface of the cryogenic liquid. In contrast to SFL, the URF particle engineering process applied in the present study utilizes rapid freezing of a drug/excipient solution onto a cryogenic substrate of desired thermal conductivity to obtain a solid dispersion/solution without triggering the Liedenfrost effect. Therefore, URF does not present the problems associated with SFL, such as recovering the particles from the cryogenic liquid, handling the cryogenic liquid, triggering the Liedenfrost effect and environmental issues.

FIG. 2 illustrates processing by URF, showing the scraper plate 10, the feed solution 12, the rotating drum 14 cooled by liquid nitrogen to −80° C., the frozen feed solution 16, the collector 18 filled with liquid nitrogen, frozen particles 20, lyophilizer 22, and dry powder 24. The composition compositions of Dry Powders (by weight): ITZ/PVP═ITZ:lecithin:PVP 2:1:1, ITZ/PVA=ITZ:lecithin:PVA 2:1:1, ITZ/HPMC═ITZ:lecithin:HPMC 2:1:1, and ITZ potency=50% w/w.

The ITZ samples generated using the O/W template emulsion system and the co-solvent system samples were processed by URF using the apparatus shown in FIG. 2. Samples were fed as discrete droplets onto a chilled rotating drum maintained at approximately −80° C. The frozen material was removed from the drum by a scraper blade, collected, and dried using a Virtis Advantage top tray lyophilizer (The VirTis Company, Inc., Gardiner, N.Y.).

The URF-processed dry powders containing ITZ, lecithin, and a hydrophilic polymer excipient, were designated as ITZ/PVP, ITZ/HPMC or ITZ/PVA according to the hydrophilic polymer excipient used.

(3) Emulsion characterization: Droplet size measurements of the emulsion feed dispersion prior the URF processing were conducted by low angle laser light scattering using a Malvern Mastersizer S (Malvern Instruments Limited, Worcestershire, UK).

(4) Powder characterization: The following techniques were used to characterize the URF-processed dry powders: (i) Scanning Electron Microscopy: Scanning electron microscopy (SEM) was conducted using a LEO 1530 scanning electron microscope (Carl Zeiss SMT, Peabody, Mass., USA) operated at an accelerating voltage of 10 kV; (ii) Specific Surface Area: Brunauer-Emmett-Teller (BET) specific surface area measurements were performed using a Nova 2000 Version 6.11 instrument with Nova Enhanced Data Reduction Software version 2.13 (Quantachrome Corporation, Boynton Beach, Fla.) using nitrogen as the adsorbate gas; (iii) Powder X-Ray Diffraction: Powder x-ray diffraction (PXRD) was performed using a Philips 1710 X-ray diffractometer (Philips Electronic Instruments, Mahwah, N.J.); (iv) X-ray Photoelectron Spectroscopy: Determination of surface elemental compositions of powders was conducted by X-ray photoelectron spectroscopy (XPS) using an AXIS HS photoelectron spectrometer with a monochromatic Al Ka X-ray source (Kratos Analytical, Manchester, UK); and (v) Dissolution Testing at Supersaturated Conditions: Dissolution testing at supersaturated conditions was conducted in a United States Pharmacopeia (USP) 29 dissolution apparatus model Vankel 7010 Dissolution Tester (Vankel Technology Group, Cary, N.C.) at pH 1.2 using 100 mL glass dissolution vessels and stirred with small paddles at 100 rpm, temperature 37.5±0.2° C.; the amount of powder employed equaled to 10× and 100× the equilibrium solubility of crystalline ITZ at pH 1.2 (C_{eq}=5 μg/mL); 1 mL samples were collected at predetermined time points (n=3).

The URF process was employed to make nanostructured powders with an ITZ potency of 50% w/v. The ITZ:lecithin:hydrophilic polymer composition of the dry powders, wherein the hydrophilic polymers used were polyvinyl pyrrolidone PLASDONE® K17 (PVP), polyvinyl alcohol (PVA), and hydroxypropyl methylcellulose E5 (HPMC), was 2:1:1 by weight in every case (FIG. 2).

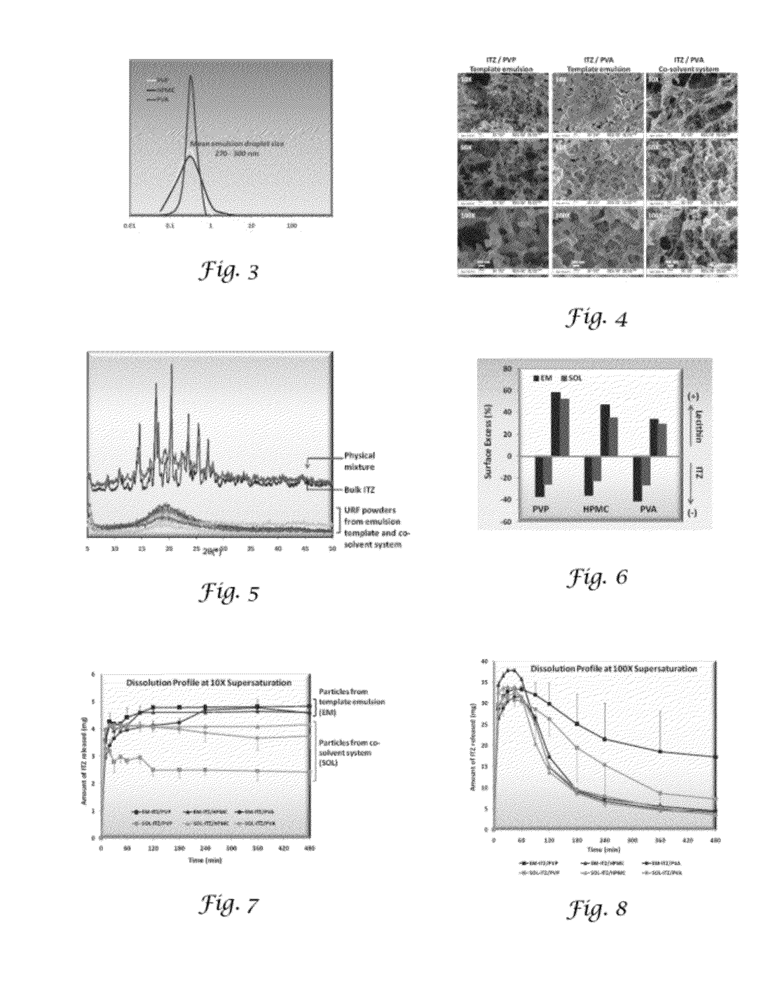

(1) Template Emulsion Droplet Sizes: Particle size distribution, based on volume fraction, was measured by laser diffraction (FIG. 3). Mean emulsion droplet sizes for ITZ:lecithin:PVP, ITZ:lecithin:HPMC, and ITZ:lecithin:PVA were between 270 and 300 nm. ITZ:lecithin:PVP droplets were between 0.157 and 0.390 μm, with a mean size of 0.270 μm. ITZ:lecithin:HMPC droplets were between 0.103 and 0.663 min, with a mean size of 0.270 μm. ITZ:lecithin:PVA droplets were between 0.207 and 0.453 μm, with a mean size of 0.300 μm (TABLE A). The distribution of submicron droplets was found to be narrow, as indicated by the span indexes range between 0.8 and 2.057.

TABLE 1
Droplet sizes of emulsion template formulations

| | Size (μm) | | | | Max C.V. |
|---|---|---|---|---|---|
| | D (0.1) | D (0.5) | D (0.9) | Span | (%) |
| ITZ/PVP | 0.157 ± 0.006 | 0.270 ± 0.000 | 0.390 ± 0.000 | 0.862 ± 0.025 | 3.69 |
| (ITZ:lecithin:PVP = 2:1:1) | | | | | |
| EXAMPLE 1 | | | | | |
| ITZ/PVA | 0.207 ± 0.006 | 0.300 ± 0.000 | 0.453 ± 0.000 | 0.800 ± 0.049 | 6.06 |
| (ITZ:lecithin:PVA = 2:1:1) | | | | | |
| EXAMPLE 2 | | | | | |
| ITZ/HPMC | 0.103 ± 0.006 | 0.270 ± 0.000 | 0.663 ± 0.006 | 2.057 ± 0.006 | 5.59 |
| (ITZ:lecithin:HPMC = 2:1:1) | | | | | |
| EXAMPLE 3 | | | | | |

(2) Dry Powder Morphology: SEM was used to evaluate the morphology of the URF-processed dry powder samples. SEM micrographs show a highly porous nanostructured aggregate structure and submicron primary domains. Fine emulsion droplets processed by URF served as template for the formation of micron-size aggregates and submicron primary particles (FIG. 4). Solid dispersions or solid solutions of poorly water-soluble drugs have greatly enhanced extents and rates of dissolution, due to increased exposure area of drug to the dissolution media and higher Gibbs free energy of the amorphous versus crystalline states. The highly porous structures shown in FIG. 4 provide a large surface area with potential increased dissolution rates both in vitro and in vivo. This would lead to significantly improved bioavailability, and therefore, is of interest to pharmaceutical formulation scientists.

(3) Specific Surface Area: The specific surface area of URF-processed formulations was 14.9 m²/g for ITZ/PVP (ITZ:lecithin:PVP), 25.6 m²/g for ITZ/HPMC (ITZ:lecithin:HPMC), and 36.7 m²/g for ITZ/PVA (ITZ:lecithin:PVA), in contrast to 4.22 m²/g for the unprocessed bulk ITZ (TABLE 2). The URF process rendered the URF-processed powders 4-9 times greater surface area as compared to that of the bulk crystalline ITZ.

TABLE 2
Specific surface areas of URF (Thin Film
Freezing)-processed powder compositions.

Specific surface area (m²/g)

| | | |
|---|---|---|
| | Emulsion | Co-Solvent |
| Formulation | Template | Formulation |

| | |
|---|---|
| Crystalline ITZ | 4.22 |

| | | |
|---|---|---|
| ITZ/PVP | 14.9 | 17.3 |
| (ITZ:lecithin:PVP K17 = 2:1:1) | | |
| ITZ/PVA | 36.7 | 23.5 |
| (ITZ:lecithin:PVA = 2:1:1) | | |
| ITZ/HPMC | 25.6 | 26.0 |
| (ITZ:lecithin:HPMC E5 = 2:1:1) | | |

(4) X-ray Diffractogram of ITZ and URF powders: ITZ is a highly crystalline hydrophobic molecule with a molecular weight of 705.64. The degree of crystallinity in ITZ/excipient mixtures has been previously shown to affect the solubility and dissolution rate of ITZ in the mixture (Vaughn et al., 2005). The degree of crystallinity of bulk ITZ, URF-processed powders, and the physical mixture were examined by X-ray diffraction and the profiles are depicted in FIG. 5. The diffractogram of bulk ITZ and physical mixture shows that the samples are highly crystalline, with intense peaks between 14 and 25° (2 θ) (peaks located at 14.4°, 17.5°, 20.4°, 23.4°, 25.3°, and 27.1°). The physical mixtures of ITZ:lecithin:hydrophilic polymers showed a quantitative reduction in crystalline intensity. The diffractogram shows amorphous halo patterns for the URF-processed powders, indicating amorphous character (ITZ in molecular dispersion within the excipient matrices) (FIG. 5).

(5) X-ray Photoelectron Spectroscopy: This technique was used to determine the elemental composition of the particle's surface. There is a negative surface excess of ITZ and a positive surface excess of lecithin in the URF-processed powders (TABLE 3). This suggests a preferential arrangement of lecithin on the surface of URF-engineered particles. This arrangement can be attributed to the aqueous external environment of both the emulsion template and co-solvent systems (See FIG. 6 and TABLE 3).

Surface ITZ is 12-15% lower in particles from the emulsion template system than in particles from the co-solvent system. Conversely, surface lecithin is 4-12% higher in particles from the emulsion template system than in particles from the co-solvent system.

Application of the O/W emulsion template method followed by URF processing resulted in reduced ITZ and increased lecithin distribution on particle surface due to arrangement of lecithin molecules at the oil-aqueous interface surrounding of emulsion droplets containing ITZ. The hydrophilic polymer molecules were also located at the vicinity of the emulsion droplets. In co-solvent systems, adsorption of lecithin and polymer molecules on ITZ was largely random and less concentrated.

TABLE 3
Surface elemental composition and surface excess of URF-processed (Thin Film Freezing-
processed) powders from template emulsion (EM) and control formulation (SOL)

| | Mass Concentration (%) | | | | Surface composition (%) ^{c} | | | | Surface excess (%) ^{d} | | | |
|---|---|---|---|---|---|---|---|---|---|---|---|---|
| | Chlorine ^{a} | | Phosphorus ^{b} | | ITZ | | Lecithin | | ITZ | | Lecithin | |
| | EM ^{e} | SOL ^{e} | EM | SOL | EM | SOL | EM | SOL | EM | SOL | EM | SOL |
| ITZ/PVP | 1.22 | 2.33 | 2.74 | 2.55 | 12.8 | 24.4 | 83.0 | 77.3 | −37.2 | −25.6 | 58.0 | 52.3 |
| ITZ/HPMC | 1.33 | 2.60 | 2.38 | 1.98 | 13.9 | 27.3 | 72.1 | 60.0 | −36.1 | −22.7 | 47.1 | 35.0 |
| ITZ/PVA | 0.83 | 2.22 | 1.94 | 1.80 | 8.7 | 23.3 | 58.8 | 54.5 | −41.3 | −26.7 | 33.8 | 29.5 |
| ITZ | 9.54 | 9.54 | | | 100.0 | 100.0 | | | | | | |
| Lecithin | | | 3.30 | 3.30 | | | 100.0 | 100.0 | | | | |

^{a} ITZ composition is represented by the chlorine atom unique to the ITZ molecule;
^{b} Lecithin composition is represented by the phosphorus atom unique to the lecithin molecule;
^{c} Normalized to mass concentration of pure ITZ and lecithin;
^{d} Based on the formulation ratio of ITZ:lecithin:polymer of 2:1:1, the theoretical composition ITZ is 50% and lecithin is 25% for powders with homogenous distribution of all components present in the formulation;
^{e} EM = emulsion template, SOL = co-solvent system.

(6) Supersaturated dissolution testing: To assess the performance of developmental compositions prior to animal testing, in vitro dissolution has routinely been used in the pharmaceutical industry. Dissolution studies reported in the literature and also testing recommended by the Food and Drug Administration have generally been conducted under sink conditions, wherein the concentrations are maintained at least three to five times below equilibrium solubility. Numerous articles have correlated the results of these tests to the in vivo performance of the formulations; however, with amorphous compositions these tests neglect the ability of the formulation to supersaturate the dissolution media. Supersaturation can occur in vivo as well, necessitating the requirement for evaluation of the associated dissolution kinetics. For this study, the dissolution testing was conducted in the present study under supersaturated conditions in order to evaluate the supersaturation dynamics of URF-processed ITZ complexes.

The maximum concentration of dissolved ITZ was determined under supersaturated conditions (10× C_{eq} and 100× C_{eq}). The results are shown in FIG. 7 for 10× and in FIG. 8 for 100× C_{eq}. URF-engineered particles exhibited very rapid wetting and dissolution in aqueous media, reflecting the formation of ITZ-excipient solid dispersions possessing submicron primary particles with high surface area and stabilized amorphous domains. At 10× supersaturation, precipitation of ITZ was not apparent from the dissolution profiles except for SOL-ITZ/PVP. At 100× supersaturation, ITZ release profile occurred in 2 phases, namely the rapid supersaturation phase (<1 h) and precipitation phase (>1 h until 8 h). Particles produced from emulsion templates displayed higher ITZ release in 10× supersaturated dissolution studies: 91%-97% (EM) vs. 48%-83% (SOL).

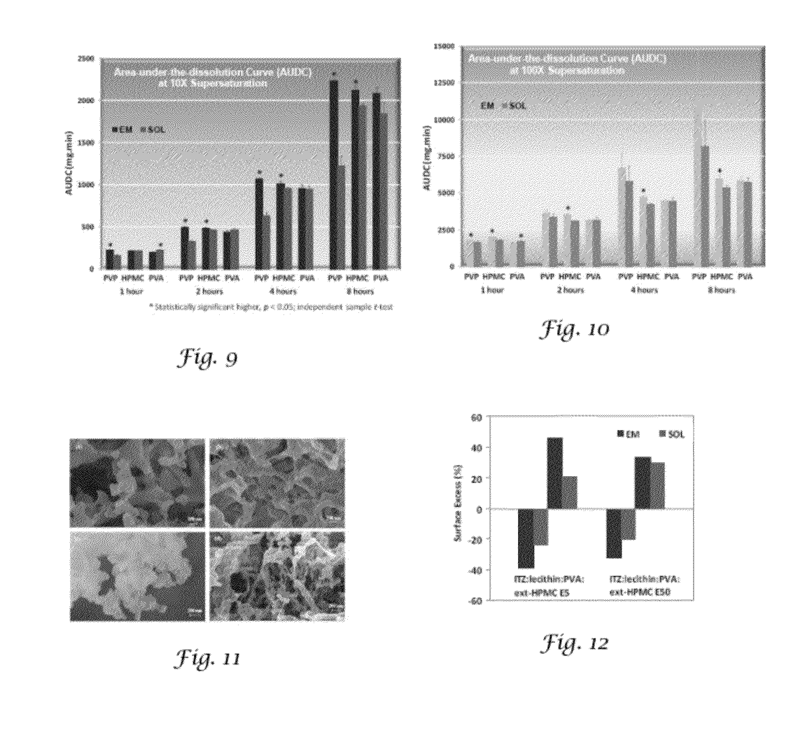

To place the results in further perspective, the extent of ITZ supersaturation was calculated as the area under the dissolution curve (AUDC) (Miller et al., 2008) (See FIG. 9 and FIG. 10). At 10× supersaturation, AUDC was significantly greater (p<0.05) for the URF-processed emulsion template samples (EM) than for the URF-processed co-solvent samples (SOL) from 2 h onwards. The largest effect was observed for particles produced from emulsion templates where PVP was the hydrophilic polymer used (i.e., the ITZ/PVP formulation). In contrast, at 100× supersaturation, significantly higher AUDC occurred only at short time (1 h). At 100× supersaturation, higher concentration level and AUDC of ITZ/PVP (similarly to the effect observed at 10× supersaturation); however, this trend was not statistically significant due to high variability associated with the ITZ/PVP formulation.

Particles produced by emulsion templating followed by URF demonstrated better wetting and more rapid supersaturation due to preferential arrangement of lecithin and hydrophilic polymer molecules on the surface ITZ particles. This is indicated by lower surface excess of ITZ and higher surface excess of lecithin in emulsion templated particles. However, the advantage of the greater surface coverage of ITZ with lecithin and hydrophilic polymers was largely negated at very high ITZ supersaturation (100×) because precipitation of ITZ predominated. Thus, additional stabilizing excipient is needed in the formulations in order to prevent or slow down precipitation.

Conclusions: Template emulsions and co-solvent systems were successfully used with the URF process for engineering micronized ITZ particles with high surface area and enhanced dissolution. The emulsion templating approach was more effective in producing ITZ particles with rapid wetting and increased extent of dissolution as compared to the co-solvent approach.

As used herein, emulsifying agents are those agents capable of enriching surface of cryogenically processed particles, so some agents may be included that do not have an effect on surface tension, i.e. hydrophilic polymers like HPMC, HPC.

### Enabling Examples

The following terms are used in the subsequent examples: “ITZ” is itraconazole, “TFF” is thin film freezing or URF, ultra rapid freezing, “PVP” is polyvinylpyrrolidone, Plasdone® K17, “PVA” is polyvinylalcohol (hydrolyzed), “HPMC E5” is hydroxypropylmethylcellulose, Methocel® HPMC E5.

### Example 1

Aliquots of 1.0 g of ITZ and 0.5 g lecithin were dissolved in 10 mL chloroform which served as the organic phase. An aliquot of 0.5 g PVP was dissolved in 40 mL of deionized water which served as the aqueous phase. The aqueous phase was gently poured into the glass container holding the organic phase to form an aqueous layer above the organic phase. The tip of a probe sonicator (Branson Sonifier® A-450A, Branson, Danbury, Conn., USA) was gently lowered into the aqueous-organic interface and the liquid mixture was sonicated for 5 min to obtain a oil-in-water emulsion. The temperature of the emulsion was maintained between 15° C. and 20° C. using a water bath throughout the sonication process. The emulsion was applied as discrete droplets onto the cryogenic rotating drum of the TFF apparatus maintained at approximately −80° C. The droplets were deformed into thin films or splats and immediately frozen on impact with the cryogenic drum. The frozen materials were removed from the drum by a scraper blade, collected in a glass container filled with liquid nitrogen and immediately lyophilized in a tray lyophilizer (Virtis Advantage, The VirTis Company, Inc., Gardiner, N.Y., USA) to obtain the dry powder. The ITZ potency in the dry powder was 50%.

### Example 2

Aliquots of 1.0 g of ITZ and 0.5 g lecithin were dissolved in 10 mL chloroform which served as the organic phase. An aliquot of 0.5 g PVA was dissolved in 40 mL of deionized water which served as the aqueous phase. The aqueous phase was gently poured into the glass container holding the organic phase to form an aqueous layer above the organic phase. The tip of a probe sonicator (Branson Sonifier® A-450A, Branson, Danbury, Conn., USA) was gently lowered into the aqueous-organic interface and the liquid mixture was sonicated for 5 min to obtain a oil-in-water emulsion. The temperature of the emulsion was maintained between 15° C. and 20° C. using a water bath throughout the sonication process. The emulsion was applied as discrete droplets onto the cryogenic rotating drum of the TFF apparatus maintained at approximately −80° C. The droplets were deformed into thin films or splats and immediately frozen on impact with the cryogenic drum. The frozen materials were removed from the drum by a scraper blade, collected in a glass container filled with liquid nitrogen and immediately lyophilized in a tray lyophilizer (Virtis Advantage, The VirTis Company, Inc., Gardiner, N.Y., USA) to obtain the dry powder. The ITZ potency in the dry powder was 50%.

### Example 3

Aliquots of 1.0 g of ITZ and 0.5 g lecithin were dissolved in 10 mL chloroform which served as the organic phase. An aliquot of 0.5 g HPMC E5 was dissolved in 40 mL of deionized water which served as the aqueous phase. The aqueous phase was gently poured into the glass container holding the organic phase to form an aqueous layer above the organic phase. The tip of a probe sonicator (Branson Sonifier® A-450A, Branson, Danbury, Conn., USA) was gently lowered into the aqueous-organic interface and the liquid mixture was sonicated for 5 min to obtain an oil-in-water emulsion. The temperature of the emulsion was maintained between 15° C. and 20° C. using a water bath throughout the sonication process. The emulsion was applied as discrete droplets onto the cryogenic rotating drum of the TFF apparatus maintained at approximately −80° C. The droplets were deformed into thin films or splats and immediately frozen on impact with the cryogenic drum. The frozen materials were removed from the drum by a scraper blade, collected in a glass container filled with liquid nitrogen and immediately lyophilized in a tray lyophilizer (Virtis Advantage, The VirTis Company, Inc., Gardiner, N.Y., USA) to obtain the dry powder. The ITZ potency in the dry powder was 50%.

### Example 4

Aliquots of 0.8 g ITZ was dissolved in 104 mL of 1,4-dioxane, 0.4 g of lecithin was dispersed in 30 mL deionized water and 0.4 g of hydrophilic polymer was dissolved in 26 mL of deionized water. The hydrophilic polymer consisted of one of the following: PVP, PVA, or HPMC E5. The aqueous lecithin and hydrophilic polymer solutions were added to the organic ITZ solution to produce a homogenous co-solvent mixture by slow stirring using a magnetic stir bar. The co-solvent mixtures were denoted as the “control”. The control formulations were applied as discrete droplets onto the cryogenic rotating drum of the TFF apparatus maintained at approximately −80° C. The droplets were deformed into thin films or splats and immediately frozen on impact with the cryogenic drum. The frozen materials were removed from the drum by a scraper blade, collected in a glass container filled with liquid nitrogen and immediately lyophilized in a tray lyophilizer (Virtis Advantage, The VirTis Company, Inc., Gardiner, N.Y., USA) to obtain the dry powder. The ITZ potency in the dry powder was 50%.

### Example 5

Emulsions were prepared according to procedures outlined in Examples 1, 2 and 3 with the same formulations. Emulsion droplet size distributions were determined by laser light scattering using a Malvern Mastersizer-S (Malvern Instruments, Ltd., Worcestershire, UK). An appropriate amount of emulsion was dispensed into approximately 600 mL deionized water to produce a light obscuration ranging from 10% to 15%. The emulsion droplet size distributions based on volume fraction is shown in TABLE 1. The mean emulsion droplet sizes were in the submicron range of 270-300 nm indicating the presence of very fine emulsion droplets. The emulsion droplet sizes remained relatively unchanged for up to 45 min after emulsion production by sonication indicating that the emulsion formulations remained stable throughout the duration of processing by TFF.

### Example 6

Powders containing ITZ were prepared according to procedures outlined in Examples 1, 2, 3, and 4 with the same formulations. Particle morphology of the powders were visualized using a scanning electron microscope (LEO 1530, Carl Zeiss SMT, Peabody, Mass., USA) operated at an accelerating voltage of 10 kV. The powders were mounted on aluminum stages using double sided carbon tape. The powders sputter coated by platinum for 30 s. Scanning electron micrographs demonstrated highly porous, nano structured aggregates with submicron primary domains (FIG. 11).

### Example 7

Powders containing ITZ were prepared according to procedures outlined in Examples 1, 2, 3, and 4 with the same formulations. Specific surface areas of the powders were measured using a Nova 2000 v.6.11 instrument (Quantachrome Instruments, Boynton Beach, Fla., USA) with nitrogen adsorbate gas. An accurately weighed amount of powder of approximately 0.25 g was degassed in the sample cell for about 12 to 18 hours prior to analysis. The specific surface area, defined as surface area per gram of sample was measured using a six-point pressure profile and quantified based on the Brunauer, Emmett, and Teller model using the Nova Enhanced Data Reduction Software v.2.13. The specific surface area of TFF-processed powders from emulsion formulations (Examples 1, 2, and 3) and control formulations (Examples 4) were presented in TABLE 2. The specific surface areas if all TFF-processed powders were at least 3.5-fold higher than the bulk crystalline ITZ. The powder originated from the emulsion formulation of ITZ:lecithin:HPMC E5=2:1:1 demonstrated higher specific surface area than the corresponding control formulation highlighting the benefit of emulsion template method in particle engineering with the TFF process.

### Example 8

Powders containing ITZ were prepared according to procedures outlined in Examples 1, 2, 3, and 4 with the same formulations. The physical mixture was prepared by co-grinding ITZ, lecithin and HPMC E5 in a ratio of 2:1:1 using a mortar and pestle. X-ray diffraction analyses were performed to evaluate the degree of crystallinity of the TFF-processed powders, physical mixture and bulk crystalline ITZ using a Philips 1710 X-ray diffractometer (Philips Electronic Instruments, Mahwah, N.J.). Sample was filled into the sample holder and a slight pressure was applied on the surface to obtain a flat powder bed of approximately 1 mm thick. The diffraction profile was measured from 5° to 50° using a 20 step size of 0.05° and a dwell time of 2 s. All the TFF-processed formulations were in amorphous form as demonstrated by the halo pattern and the total absence of the characteristic ITZ diffraction peaks at 20 between 14° to 27° as seen in the bulk crystalline ITZ and the co-ground physical mixture (FIG. 5). This indicated the presense of ITZ in molecular dispersion within the excipients after TFF processing.

### Example 9

Powders containing ITZ were prepared according to procedures outlined in Examples 1, 2, 3, and 4 with the same formulations. The elemental composition of the particle surfaces was determined using X-ray photoelectron spectroscopy (XPS). The XPS measurements were performed using an AXIS HS photoelectron spectrometer (Kratos Analytical Ltd., Manchester, UK) with a monochromatic Al Kα X-ray source. The powder samples were loaded into the sample holder as a flat, loosely packed bed of powders. An area of 300×700 μm and a depth of 8-10 nm were probed. TABLE 3 shows the surface elemental composition in term of mass concentration percent and surface excess of TFF-processed powders from template emulsion (EM) and control formulations (SOL). ITZ composition was represented by the chlorine atom unique to the ITZ molecule while lecithin composition is represented by the phosphorus atom unique to the lecithin molecule. The percent surface composition was obtained by normalizing the mass concentration of chlorine and phosphorus atom in each formulation to the mass concentration of pure ITZ (9.54%) and lecithin (3.30%). Based on the formulation ratio of ITZ:lecithin:polymer of 2:1:1 (polymer means PVP, PVA or HPMC E5), the theoretical composition of ITZ is 50% and lecithin is 25% if all components present in the formulations were homogenously distributed. Percent surface excess of ITZ was calculated by deducting 50% from the percent surface compositions of ITZ while percent surface excess of lecithin was calculated by deducting 25% from the percent surface compositions of lecithin. Negative signs of surface excess for ITZ indicated relative deficiency of ITZ molecules on the particle surface as compared to the theoretical proportion (50%) while positive signs of surface excess for lecithin indicated relative excess of lecithin molecules on the particle surface as compared to the theoretical proportion (25%). The surface excess values showed that the particles have internal portion rich in ITZ and external portion rich in surfactant, namely lecithin for all the TFF-processed particles. However, particles produced from template emulsions demonstrated lower surface excess of ITZ by 12-15% and higher surface excess of lecithin by 4-12% as compared to particles produced from the control formulations. FIG. 6 illustrates the difference in surface excess for particles produced from emulsion template and control formulations. This clearly demonstrates the effectiveness of template emulsion in enriching the particle surface with surfactants such as lecithin as compared to the control formulation which utilized a co-solvent drug-excipient mixture. A greater extent of particle surface enrichment with surfactant will render the surface of hydrophobic agents such as ITZ more hydrophilic and easily wettable by water. Improved surface wettability will lead to enhanced dissolution of the hydrophobic agents and consequently enhanced bioavailability upon administration to the body.

### Example 10

Powders containing ITZ were prepared according to procedures outlined in Examples 1, 2, 3, and 4 with the same formulations. The ITZ potency of the final dry powders was 50% based on the drug-excipient ratio of ITZ:lecithin:polymer=2:1:1. Powder dissolution studies of the TFF-processed particles were carried out at 10-time supersaturation with respect to the equilibrium solubility of ITZ, C_{eq}=5 μg/mL at pH 1.2. Aliquots of 10 mg of the powder samples were added to 100 mL of pH 1.2 dissolution media which had been equilibrated to 37.0±0.2° C. and were subjected to a constant stirring speed of 100 rpm. Samples of 1 mL were collected at predetermined time points (n=3). FIG. 7 shows the dissolution profiles of particles produced from template emulsion (EM) and control formulations (SOL). Higher ITZ release was demonstrated by EM (91%-97%) as compared to SOL (48%-83%) for all the formulations tested. The enhancement of dissolution of EM was attributed to better wettability of EM owing to higher extent of ITZ surface enrichment by surfactants such as lecithin in EM as illustrated in Example 9. Since dissolution of hydrophobic agents such as ITZ is often the limiting factor in determining absorption and bioavailability, enhancement of wettability and subsequent dissolution will be highly advantageous in improving bioavailability.

### Example 11

For producing the template emulsions with an additional stabilizing polymer additive, aliquots of 1.0 g of ITZ and 0.5 g lecithin were dissolved in 10 mL chloroform which served as the organic phase. An aliquot of 0.5 g PVA was dissolved in 40 mL of deionized water which served as the aqueous phase. An aliquot of 0.25 g hydrophilic polymer was dissolved in 20 mL of deionized water which served as the external stabilizing polymer additive to the emulsion (denoted herein as ext-polymer). The ext-polymer consisted of either HPMC E5 or HPMC E50. The aqueous phase containing PVA was gently poured into the glass container holding the organic phase to form an aqueous layer above the organic phase. The tip of a probe sonicator (Branson Sonifier® A-450A, Branson, Danbury, Conn., USA) was gently lowered into the aqueous-organic interface and the liquid mixture was sonicated for 5 min to obtain a oil-in-water emulsion. The ext-polymer solution containing either HPMC E5 or HPMC E50 was immediately added to the emulsion and the mixture was gently stirred for 30 s using a magnetic stirrer. For producing the control formulations with additional stabilizing polymer additives, aliquots of 0.8 g ITZ was dissolved in 104 mL of 1,4-dioxane, 0.4 g of lecithin was dispersed in 30 mL deionized water, 0.4 g of PVA was dissolved in 26 mL of deionized water and 0.2 g hydrophilic polymer (HPMC E5 or HPMC E50) was dissolved in 16 mL of deionized water. The aqueous lecithin solution, and hydrophilic polymer solutions containing PVA and an additional stabilizing polymer additive (HPMC E5 or HPMC E50) were added to the organic ITZ solution to produce a homogenous co-solvent mixture by slow stirring using a magnetic stir bar. The emulsion template and control formulations were separately processed by TFF based on the steps illustrated in Examples 1, 2, 3, and 4. The ITZ potency of the final dry powders was 44% based on the drug-excipient ratio of ITZ: lecithin:PVA:ext-polymer=2:1:1:0.5.

The elemental composition of the particle surfaces of the TFF-processed particles with an additional stabilizing polymer additive was determined using X-ray photoelectron spectroscopy (XPS) in accordance to steps illustrated in Example 9. Powder dissolution studies were carried out at 100-time supersaturation with respect to the equilibrium solubility of ITZ, C_{eq}=5 μg/mL at pH 1.2. The dissolution studies were performed using 112.5 mg aliquots of powders in accordance to the experimental conditions outlined in Example 10.

The calculations and interpretation of surface excess was undertaken in accordance to Example 9. TABLE 4 and FIG. 12 illustrate the difference in surface excess for particles produced from emulsion template (EM) and control formulations (SOL). The effectiveness of template emulsion in enriching the particle surface with surfactants such as lecithin as compared to the control formulation which utilized a co-solvent drug-excipient mixture was clearly demonstrated by the lower ITZ surface excess and higher lecithin in EM.

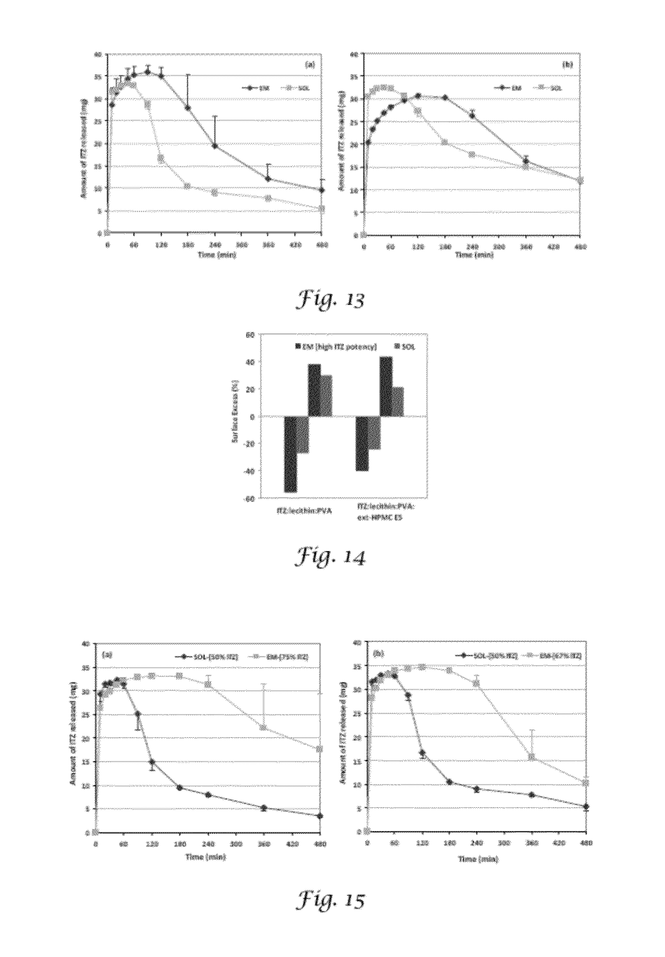

FIG. 13 shows the dissolution profiles of particles produced from EM and control SOL. The dissolution studies performed at very high ITZ supersaturation in order to evaluate the effectiveness of the additional stabilizing polymer additives in reducing the rate of ITZ precipitation in EM. Both the additional stabilizing polymer additives used, name HPMC E5 and HPMC E50 were more effective in stabilizing ITZ in EM formulations as compared to the SOL formulations. The extent of dissolution of EM was significantly higher than SOL (p<0.05, independent t-test). Extent of dissolution was represented by the total area-under-the-dissolution curve at 8-hour (AUDC), whereby total AUDC for ITZ:lecithin:PVA:ext-HPMC E5 was 10424±1625 mg.min (EM) versus 6588±234 mg min (SOL), and total AUDC for ITZ:lecithin:PVA:ext-HPMC E50 was 10903±190 mg.min (EM) versus 9709±3349 mg.min (SOL). The enhanced dissolution of EM was attributed to improved ITZ surface wettability and better ITZ protection from precipitation in view of the greater extent of surface enrichment with lecithin and hydrophilic polymers.

TABLE 4
Surface elemental composition and surface excess
of TFF-processed powders from template emulsion
(EM) and control formulations (SOL)

| | Surface excess (%) | | | |
|---|---|---|---|---|
| | ITZ | | Lecithin | |
| Formulation | EM | SOL | EM | SOL |
| ITZ:lecithin:PVA:ext-HPMC E5 | −38.9 | −24.0 | 46.3 | 20.8 |
| ITZ:lecithin:PVA:ext-HPMC E50 | −32.0 | −20.2 | 33.3 | 29.6 |

### Example 12

Powders containing higher potencies of ITZ were prepared. Powders consisting of ITZ:lecithin:PVA=6:1:1 (ITZ potency 75%) were produced from template emulsion according to procedures outlined in Example 2. Powders consisting of ITZ:lecithin:PVA=2:1:1 (ITZ potency 50%) were produced from control formulation according to procedures outlined in Example 4. Powders with additional stabilizing polymer additive consisting of ITZ:lecithin:PVA:ext-HPMC E5=6:1:1:1 (ITZ potency 67%) was produced from template emulsion and ITZ:lecithin:PVA:ext-HPMC E5=2:1:1:0.5 (ITZ potency 44%) was produced from control formulations according to procedures outlined in Example 11.

The elemental composition of the particle surfaces of the TFF-processed particles was determined using X-ray photoelectron spectroscopy (XPS) in accordance to steps illustrated in Example 9. Powder dissolution studies were carried out at 100-time supersaturation with respect to the equilibrium solubility of ITZ, C_{eq}=5 μg/mL at pH 1.2. The dissolution studies were performed in accordance to the experimental conditions outlined in Example 10 using aliquots of powders giving an equivalent amount of ITZ of 50 mg.

The calculations and interpretation of surface excess was undertaken in accordance to Example 9. TABLE 5 and FIG. 14 illustrate the difference in surface excess for particles produced from emulsion template (EM) and control formulations (SOL). The effectiveness of template emulsion containing higher potency of ITZ (75% and 67%) in enriching the particle surface with surfactants such as lecithin as compared to the control formulation (ITZ potency 50% and 44%) which utilized a co-solvent drug-excipient mixture was clearly demonstrated by the relatively lower ITZ surface excess and higher lecithin in EM. Surface enrichment of EM particles with surfactants such as lecithin still occurred despite the higher ITZ potency owing to the presence of excess lecithin in the formulations as well as to the preferential arrangement and concentration of lecithin molecules at the aqueous-organic interfaces the ITZ-rich emulsion droplets.

FIG. 15 shows the dissolution profiles of particles produced from EM and control SOL which demonstrated better dissolution for EM formulations with high ITZ potency as compare to the control formulations (SOL). The extent of dissolution of EM was significantly higher than SOL (p<0.05, independent t-test). Extent of dissolution was represented by the total area-under-the-dissolution curve at 8-hour (AUDC), whereby total AUDC for ITZ:lecithin:PVA was 13107±1894 mg.min (EM-[75% ITZ]) versus 5734±329 mg.min (SOL-[50% ITZ]), and total AUDC for ITZ:lecithin:PVA:ext-HPMC E5 was 12168±906 mg.min (EM-[65% ITZ]) versus 6588±234 mg.min (SOL-[44% ITZ]). The enhanced dissolution of EM was attributed to improved ITZ surface wettability and better ITZ protection from precipitation in view of the greater extent of surface enrichment with lecithin and hydrophilic polymers. This example shows that potency of ITZ could be significantly increased while maintaining relatively high extent of ITZ surface enrichment with surfactant and dissolution.

TABLE 5
Surface elemental composition and surface excess of
TFF-processed powders from template emulsion (EM) with
high ITZ potency and control formulations (SOL)

| | Surface excess (%) | | | |
|---|---|---|---|---|
| | ITZ | | Lecithin | |
| | EM | | EM | |
| | [high ITZ | | [high ITZ | |
| Formulation | potency] | SOL | potency] | SOL |
| ITZ:lecithin:PVA | −55.9 | −26.7 | 38.1 | 29.5 |
| ITZ:lecithin:PVA:ext-HPMC E5 | −39.9 | −24.0 | 43.1 | 20.8 |

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Chow K. T., Yang W., and Williams III R. O. (2008). Dissolution enhancement of itraconazole prepared by a modified spray freezing into liquid process with template emulsion. AAPS Journal. 10(S2): 1284.
2. Miller D. A., DiNunzio J. C., Yang W., McGinity J. W., and Williams R. O. (2008). Targeted intestinal delivery of supersaturated itraconazole for improved oral absorption. Pharm. Res. 25, 6, 1450-1459.
3. Overhoff K. A., Engstrom J. D., Chen B., Scherzer B. D., Milner T. E., Johnston K. P., and Williams III R. O. (2007). Novel ultra-rapid freezing particle engineering process for enhancement of dissolution rates of poorly water soluble drugs. Eur. J. Pharm. Biopharm., 65, 57-67.
4. Overhoff K. A. (2006) Improved Oral Bioavailability of Poorly Water Soluble Drugs Using Rapid Freezing Processes. University of Texas at Austin [Ph.D. Dissertation].
5. Rogers T. L., Overhoff K. A., Shah P., Santiago P., Yacaman M. J., Johnston K. P., and Williams R. O. (2003). Micronized powders of a poorly water soluble drug produced by a spray-freezing into liquid-emulsion. Eur. J. Pharm. Biopharm. 55, 161-172.
6. Oyewumi M. O., and Mumper R. J. (2002) Gadolinium-loaded nanoparticles engineered from microemulsion templates. Drug Dev. Ind. Pharm. 28(3), 317-328.

7. Vaughn J. M., Gao X., Yacaman M. J., Johnston K. P., and Williams 3rd R. O. (2005) Comparison of powder produced by evaporative precipitation into aqueous solution (EPAS) and spray freezing into liquid (SFL) technologies using novel Z-contrast STEM and complementary techniques. Eur. J. Pharm. Biopharm. 60, 81-89.

1. A method of making particles with surface enriched hydrophilicity by template emulsion comprising:
dissolving or dispersing one or more hydrophobic agents in an effective amount of an organic solvent and an emulsifying agent, wherein the one or more agents and the solvent form an organic phase mixture;
homogenizing the organic phase mixture with an aqueous phase mixture, to form a template emulsion; and
cryogenically processing droplets of the template emulsion by ultra rapid freezing under conditions that do not trigger a Liedenfrost effect during the freezing process to produce frozen emulsion particles.

2. The method of claim 1, wherein the template emulsion droplets are frozen in less than about 10 seconds, about 5 seconds, about 1 second or about 0.5 seconds, when contacting the cryogenic surface.

3. (canceled)

4. (canceled)

5. The method of claim 1, further comprising the step of drying the frozen emulsion particles, wherein the resulting dry powder is surface enriched for the hydrophilic excipient over the agent.

6. (canceled)

7. The method of claim 1, wherein the organic solvent in the organic phase mixture comprises one or more organic compounds and one or more emulsifying agents.

8. The method of claim 7, wherein the one or more organic compounds are defined further as organic solvents that are not miscible with a continuous external phase of emulsion.

9. The method of claim 8, wherein the one or more organic compounds comprise chloroform in the organic phase mixture at about 20% v/v.

10. The method of claim 7, wherein the one or more emulsifying agents in the organic phase mixture comprises lecithin.

11. The method of claim 1, wherein the aqueous phase mixture comprises one or more polar solvents and one or more excipients.

12. The method of claim 11, wherein the one or more polar solvents in the aqueous phase mixture comprise water.

13. The method of claim 12, wherein the concentration of water in the aqueous phase mixture is 80% v/v.

14. The method of claim 11, wherein the one or more excipients in the aqueous phase mixture comprises at least one of a hydrophilic polymer and an emulsifying agent.

15. The method of claim 14, wherein the hydrophilic polymer is at least one of a polyvinyl pyrrolidone (PVP), a polyvinyl alcohol (PVA) or a hydroxypropyl methylcellulose (HPMC).

16. The method of claim 1, wherein at least one of the one or more agents comprises an active pharmaceutical agent.

17. The method of claim 1, wherein the organic phase mixture comprises an oil.

18. The method of claim 11, wherein the one or more excipients in the aqueous phase mixture comprise a surfactant.

19. The method of claim 1, wherein at least one of the one or more agents is hydrophobic or poorly soluble in water.

20. (canceled)

21. The method of claim 20, wherein the active pharmaceutical agent is a Biopharmaceuticals Classification System (BCS) Class II or Class IV drug.

22. The method of claim 1, wherein the agent is a pharmaceutical, nutraceutical, agricultural, or veterinary product.

23. The method of claim 1, wherein the template emulsion is at least one or a single emulsion a multiple emlusion or a template emulsion is capable of remaining as an emulsion during application to the cryogenic surface of the thin film freezing apparatus.

24. (canceled)

25. (canceled)

26. The method of claim 5, wherein the powder resulting from drying the frozen emulsion particles is surface enriched such that the active composition displays a surface excess of the one or more hydrophilic excipient by X-ray photoelectron spectroscopy or another suitable method that measures surface excess of the one or more agents.

27. The method of claim 26, wherein the surface excess is greater than about 2%.

28. The method of claim 1, wherein the admixture of organic and aqueous phase mixtures is homogenized by at least one of high-shearing or ultrasonication.

29. (canceled)

30. (canceled)

31. The method of claim 1, wherein the mean template emulsion droplet size is 270-300 nm.

32. A composition made by a process comprising:
dissolving or dispersing one or more hydrophobic agents in an effective amount of an organic solvent and an emulsifying agent, wherein the one or more agents and the solvent form an organic phase mixture;
homogenizing the organic phase mixture with an aqueous phase mixture, to form a template emulsion; and
cryogenically processing droplets of the template emulsion by ultra rapid freezing under conditions that do not trigger a Liedenfrost effect during the freezing process to produce frozen emulsion particles.

33. The composition of claim 32, wherein the template emulsion droplets are frozen in less than about 10 seconds, about 5 seconds, about 1 second or about 0.5 seconds, when contacting the cryogenic surface.

34. The composition of claim 32, further comprising collecting the frozen emulsion particles.

35. (canceled)

36. (canceled)

37. (canceled)

38. The composition of claim 32, wherein the solvent in the organic phase mixture comprises one or more organic compounds and one or more emulsifying agents.

39. The composition of claim 38, wherein the one or more organic compounds are defined further as organic solvents that are not miscible with a continuous external phase of emulsion.

40. The composition of claim 39, wherein the one or more organic compounds comprise chloroform in the organic phase mixture at about 20% v/v.

41. The composition of claim 38, wherein the one or more emulsifying agents in the organic phase mixture comprise lecithin.

42. The composition of claim 32, wherein the aqueous phase mixture comprises one or more polar solvents and one or more excipients.

43. The composition of claim 42, wherein the one or more polar solvents in the aqueous phase mixture comprise water.

44. The composition of claim 43, wherein the concentration of water in the aqueous phase mixture is 80% v/v.

45. The composition of claim 42, wherein the one or more excipients in the aqueous phase mixture comprises at least one of a hydrophilic polymer and an emulsifying agent.

46. The composition of claim 45, wherein the hydrophilic polymer comprises at least one of a polyvinyl pyrrolidone, a polyvinyl alcohol (PVA) or a hydroxypropyl methylcellulose (HPMC).

47. The composition of claim 43, wherein the one or more excipients in the aqueous phase mixture comprise a surfactant.

48. The composition of claim 32, wherein at least one of the one or more agents is hydrophobic or poorly soluble in water.

49. The composition of claim 32, wherein at least one of the one or more agents is an active pharmaceutical agent.

50. The composition of claim 49, wherein the active pharmaceutical agent is a BCS Class II or Class IV drug.

51. The composition of claim 32, wherein the agent is a pharmaceutical, nutraceutical, agricultural, or veterinary product.

52. The composition of claim 32, wherein the template emulsion is at least one or a single emulsion a multiple emulsion or a template emulsion is capable of remaining as an emulsion during application to the cryogenic surface of the thin film freezing apparatus.

53. (canceled)

54. (canceled)

55. The composition of claim 36, wherein the powder resulting from drying the frozen emulsion particles is surface enriched such that the active composition displays a surface excess of the one or more hydrophilic excipients by X-ray photoelectron spectroscopy or another suitable method that measures surface excess of the one or more agents.

56. The composition of claim 55, wherein the surface excess is greater than about 2%.

57. The composition of claim 32, wherein the admixture of organic and aqueous phase mixtures is homogenized by at least one of high-shearing or ultrasonication.

58. (canceled)

59. (canceled)

60. The composition of claim 32, wherein the mean template emulsion droplet size is 270-300 nm.

61. (canceled)

62. A composition comprising:
a heterogenous lyophilized particle comprising a hydrophilic polymer having an inner portion enriched with an active ingredient and surrounded by a surface portion having a surface excess of surfactant made from a rapidly frozen homogenous solution of a template emulsion.

63. (canceled)

64. A non-encapsulated particle comprising:
a heterogenous lyophilized particle comprising a hydrophilic polymer having an inner portion enriched with an active ingredient and surrounded by a surface portion having a surface excess of surfactant made from a rapidly frozen homogenous solution of a template emulsion.

65. (canceled)

66. A particle comprising:
a heterogenous lyophilized hydrophilic polymer particle, the particle comprising an inner portion enriched with an active ingredient over a surfactant and surrounded by a surface portion having a surface excess of surfactant over active agent made from a rapidly frozen homogenous solution of a template emulsion.

67. (canceled)
